(19) United States
(12) Patent Application Publication
(10) Pub. No.: US 2010/0217085 A1
(43) Pub. Date: Aug. 26, 2010

(54) Laryngoscope Blade

(75) Inventors: Gary R. Williams, Carlsbad, CA (US); Charles E. Doerr, Vista, CA (US)

Correspondence Address: ERIC HANSCOM, 2141 Palomar Airport Road, Suite 320, CARLSBAD, CA, 92011, US

(73) Assignee: Hartwell Medical Corporation, Carlsbad, CA (US)

(21) Appl. No.: 12/390,148

(22) Filed: Feb. 20, 2009

(51) Int. Cl. A61B 1/267

(52) U.S. Cl. 600/193

(57) ABSTRACT

A laryngoscope blade in accordance with one embodiment includes a blade portion including a tab extension, the tab extension including a tab extension slot for receiving a portion of a laryngoscope handle; and a heel coupled to the tab extension by means of a fastener, the heel including a heel slot that aligns with the tab extension slot and also receives the portion of the laryngoscope handle, wherein the heel is colored to indicate a size of the blade portion of the laryngoscope blade.

### BACKGROUND

1. Field of Invention

This invention relates to laryngoscope blades. More specifically the invention relates to disposable and reusable laryngoscope blades.

2. Description of Related Art

Laryngoscope blades are manufactured in a variety of shapes and sizes. The most common styles used throughout the world are the curved, or “Macintosh,” and straight, or “Miller,” blades. Since the creation of laryngoscope blades years ago, durable blades made from stainless steel have been the popular type of blades used by medical personnel. However, thorough cleaning and disinfection of the durable blades has been less than adequate. Due to the increased awareness of new strains of bacteria and viruses, the emphasis on infection control within the medical industry has been heightened. With this new emphasis on infection control, plastic blades were introduced to reduce cost and to make laryngoscope blades single patient use devices. The new disposable laryngoscope blades, made predominantly of plastic, reduced the cost to the medical practitioner, but the blades were susceptible to breakage during an intubation procedure. Additionally, the plastic blade would often bend, thus not allowing for good visualization of the patient's vocal cords. Due to these detrimental features of the plastic blade, disposable blades constructed of stainless steel with plastic heels were introduced. While better than the all plastic blades, the plastic heel still has the potential for breakage at the connection point of the blade to the handle.

Additionally, with the introduction of plastic blades came the introduction of plastic rods bent to form a light tube for illumination of the vocal cords. This again was a cost saving measure that resulted in an inferior light source for emergency intubation procedures. The bent clear plastic light conduit was not as bright as a true fiber optic bundle and on occasion has been falsely marketed as a disposable fiber optic laryngoscope.

Both disposable and reusable laryngoscope blades are difficult to differentiate by size under emergency conditions and particularly in the pre-hospital care environment. Most blades have a small size number on the end of the heel which is difficult to see when the blade is packaged, lying on the ground, on a surgical tray, in low light conditions or when stored in a carrying case like those used by paramedics in the field. With the introduction of molded plastic heels the size designation of the blade is sometimes molded into the plastic heel making it even harder to see.

### SUMMARY

The present embodiments overcome these and other deficiencies of laryngoscope blades by providing an improved laryngoscope blade that may include one or more of the following features: (1) a color coded plastic heel, (2) a threaded light conduit to securely fasten to a plastic heel, (3) a continuous formed piece of metal for the blade and an extended tab portion, (4) an extended tab portion of metal that includes a notch that is substantially similar to a notch in the plastic heel that attaches to a pin on a handle, (5) a hole in the extended table portion of metal such that the color coded plastic heel is visable from both sides of the laryngoscope blade, and/or (6) a marking on the blade portion that shows the size of the blade.

A laryngoscope blade in accordance with one embodiment includes a blade portion including a tab extension, the tab extension including a tab extension slot for receiving a portion of a laryngoscope handle and a heel coupled to the tab extension by means of a fastener, the heel including a heel slot that aligns with the tab extension slot and also receives a portion of the laryngoscope handle, wherein the heel is colored to indicate a size of the blade portion of the laryngoscope blade.

A set of blades in accordance with another embodiment includes a plurality of laryngoscope blades each including a blade portion and a tab extension, the tab extension including a tab extension slot for receiving a portion of a laryngoscope handle, wherein the plurality of laryngoscope blades includes blades of at least two different sizes and a plurality of heels coupled to the tab extension of each of the plurality of laryngoscope blades by means of a fastener, the heel of each of the plurality of laryngoscope blades including a heel slot that aligns with the tab extension slot, the heel slot receiving the portion of the laryngoscope handle, wherein the plurality of heels are colored to indicate the size of the laryngoscope blades.

Some embodiments include a conduit coupled to the heel, the conduit including a threaded portion that engages a threaded hole in the heel and a lamp coupled to the conduit. Optionally, some embodiments include a metal contact coupled to the heel and a wire contained at least partially within the conduit and coupled to the metal contact at a first end and coupled to the lamp at a second end. Optionally, the conduit is located on a first side of the blade portion and the lamp is positioned through a slot in the blade portion such that the distal end of the lamp is located on a second side of the blade portion of the laryngoscope blade. The laryngoscope can include a hole in the tab extension of the blade such that the heel is visible through the hole. In some embodiments, a fastener is attached to the tab extension by means of a threaded hole in the tab extension and can include spring loaded ball poppets used to connect the laryngoscope blade to a laryngoscope handle. The blade and tab extension are formed from a single piece of metal in some embodiments. The laryngoscope blade can be a disposable blade or a reusable blade.

The foregoing, and other features and advantages will be apparent from the following, more particular description of the various embodiments, the accompanying drawings, and the claims.

### BRIEF DESCRIPTION OF THE DRAWINGS

The above and other aspects, features and advantages of the various embodiments will be more apparent from the following more particular description thereof, presented in conjunction with the following drawings, wherein:

FIG. 1 is a three dimensional diagram illustrating a laryngoscope blade in accordance with one embodiment;

FIG. 2 is a top view of the laryngoscope blade of FIG. 1;

FIG. 3 is a bottom view of the laryngoscope blade of FIG. 1;

FIG. 4 is an end view of the laryngoscope blade of FIG. 1;

FIG. 5 is a side view of the laryngoscope blade of FIG. 1;

FIG. 6 is a side cross sectional view of the laryngoscope blade of FIG. 1 showing a threaded heel section in accordance with one embodiment;

FIG. 7 is a three dimensional diagram illustrating a laryngoscope blade in accordance with another embodiment;

FIG. 8 is a top view of the laryngoscope blade of FIG. 7;

FIG. 9 is a bottom view of the laryngoscope blade of FIG. 7;

FIG. 10 is an end view of the laryngoscope blade of FIG. 7;

FIG. 11 is a side view of the laryngoscope blade of FIG. 7; and

FIG. 12 is a side cross sectional view of the laryngoscope blade of FIG. 7 showing a threaded heel section in accordance with one embodiment.

Corresponding reference characters indicate corresponding components throughout the several views of the drawings. Skilled artisans will appreciate that elements in the figures are illustrated for simplicity and clarity and have not necessarily been drawn to scale. For example, the dimensions, sizing, and/or relative placement of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will also be understood that the terms and expressions used herein have the ordinary meaning as is usually accorded to such terms and expressions by those skilled in the corresponding respective areas of inquiry and study except where other specific meanings have otherwise been set forth herein.

### DETAILED DESCRIPTION OF THE DRAWINGS

The following description is not to be taken in a limiting sense, but is made merely for the purpose of describing the general principles of the present embodiments. The scope of the invention should be determined with reference to the claims. The present embodiments address the problems described in the background while also addressing other additional problems as will be seen from the following detailed description.

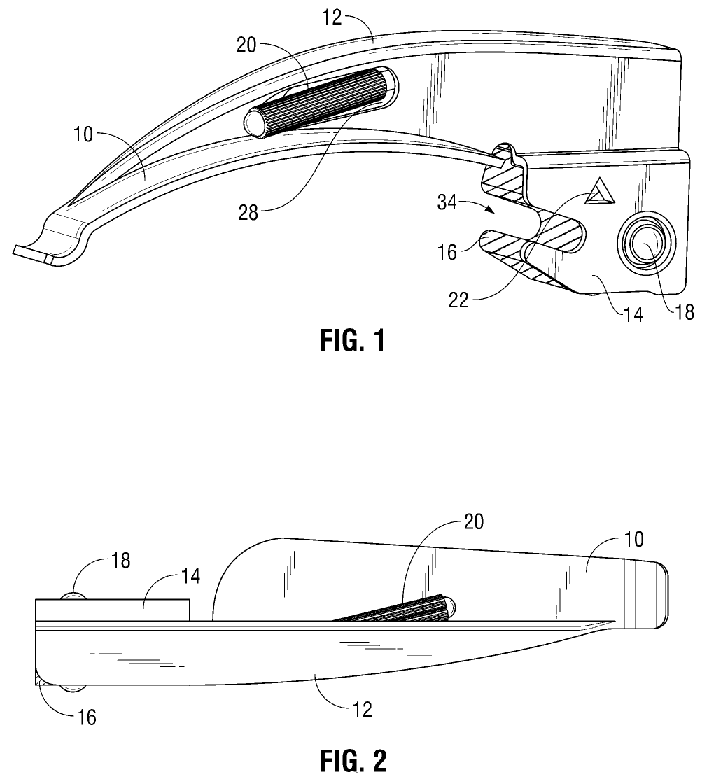

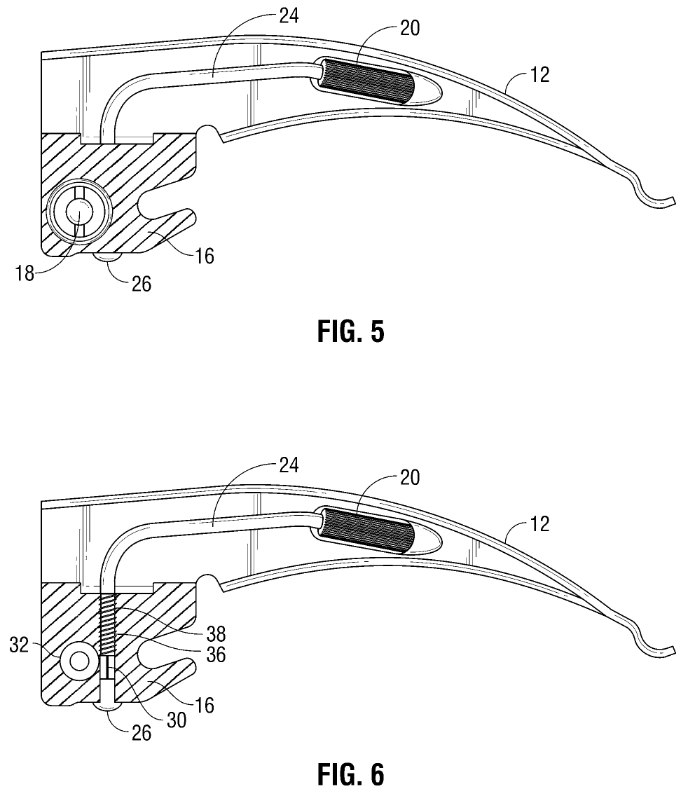

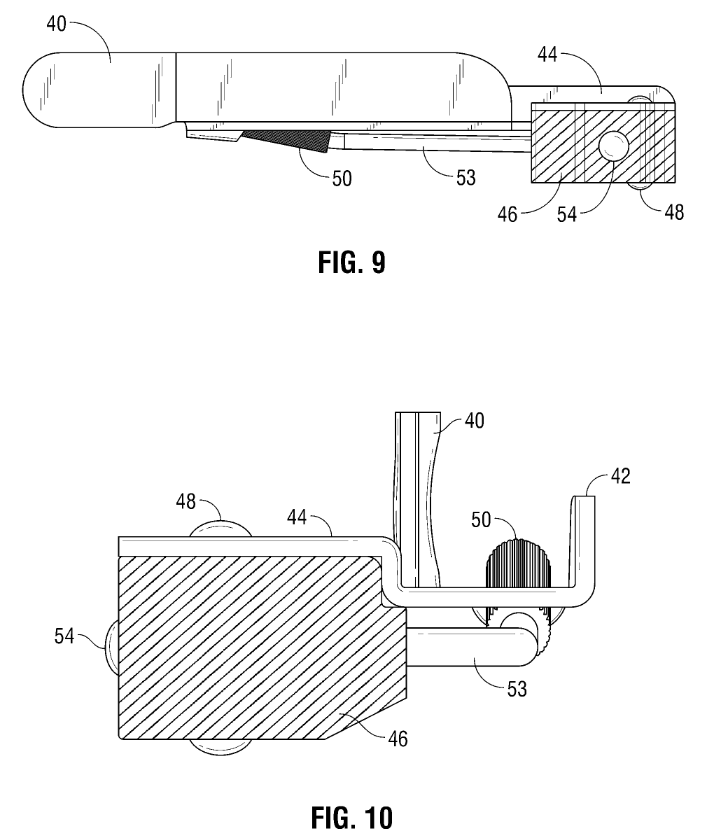

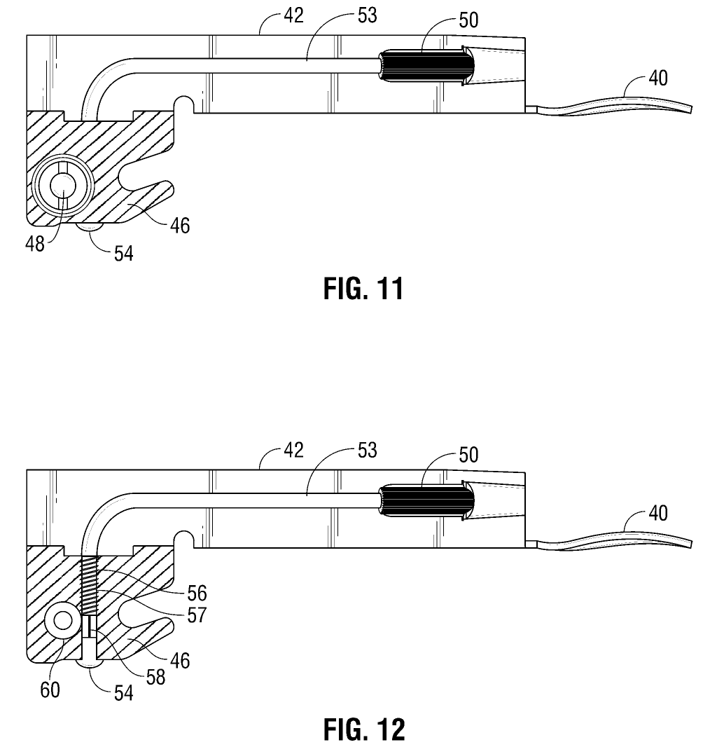

Further features and advantages, as well as the structure and operation of various embodiments, are described in detail below with reference to the accompanying FIGS. 1-12. The embodiments are described in the context of a single use (disposable) laryngoscope blade. Nonetheless, one of ordinary skill in the art readily recognizes that some or all of the features and/or advantages of the embodiments are also applicable to reusable laryngoscope blades.

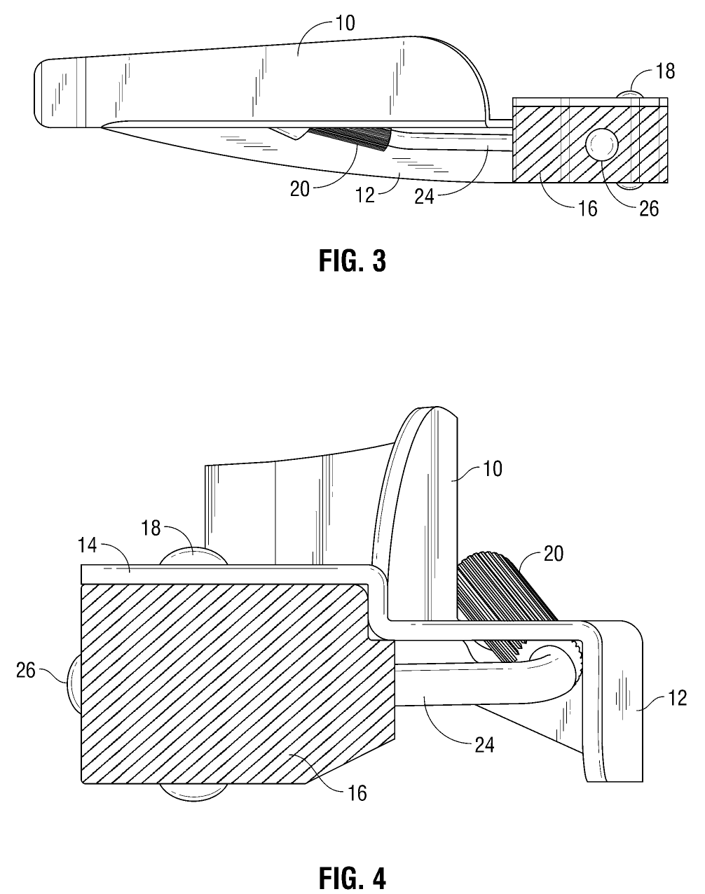

Referring to FIG. 1, a three dimensional diagram illustrates a laryngoscope blade in accordance with one embodiment. The curved laryngoscope blade shown in FIG. 1 is known as a “Macintosh” blade. Shown are a blade 10, a top portion of the blade 12, a tab extension 14 of the blade, a heel 16, a fastener 18, a lamp 20, a hole 22 in the tab extension 14 of the blade, a slot 28 in the blade, and a slot 34 in the tab extension 14 and heel 16. Referring to FIGS. 2-5, various alternative views of the laryngoscope blade of FIG. 1 are shown. Shown in FIGS. 3-5 are a conduit 24 and a metal contact 26.

The blade 10, the top portion of the blade 12 and the tab extension 14, in accordance with one embodiment are formed from a single piece of metal (e.g., stainless steel). The tab extension 14 is connected to the heel 16 by the fastener 18. The tab extension 14 and the heel 16 both include a slot 34 which is used to connect the laryngoscope blade to a laryngoscope handle (not shown) during use. Forming the blade 10, the top portion of the blade 12 and the tab extension 14 from a single piece of metal provides a strong blade that is easily manufactured. This avoids the need for welding which can potentially cause weak points in the device and adds to the overall cost of manufacturing. As will be described below in greater detail, the slot 34 in the tab extension 14 provides a reinforced metal-to-metal connection to the laryngoscope handle making the blade less susceptible to breaking away from the laryngoscope handle during use.

The laryngoscope blade further includes the lamp 20 and the conduit 24. The conduit 24 is coupled to the lamp 20 at a distal end and the heel 16 at a proximal end. In the present embodiment, the conduit 24 houses a metal wire that is coupled to the metal contact 26 at a proximal end and the lamp 20 at a distal end (shown in FIG. 6). The conduit 24 is located on a back side of the laryngoscope blade and the lamp 20 is placed through a slot 28 in the blade 10 such that the distal end of the lamp 20 is located on a front side of the laryngoscope blade. In this regard, the lamp 20 is held in place without the need to secure the lamp 20 to the blade 10, for example, through welding. As described, the laryngoscope blade is used in connection with a laryngoscope handle (not shown). The laryngoscope handle is generally reusable and can be easily attached and detached from the laryngoscope blades. In one embodiment, the laryngoscope handle includes batteries that provide power to the lamp 20 of the laryngoscope blade. The metal contact 26 provides an electrical contact between the batteries in the laryngoscope handle and the lamp 20 via the metal wire located within the conduit 24. In an alternative embodiment, the conduit 24 and the lamp 20 are replaced with a light pipe or a fiber optic bundle. In this embodiment, the light source is actually contained within the laryngoscope handle and directs light into the light pipe or fiber optic bundle.

As described, laryngoscope handles include a metal bar that slides into the slot 34 and is used to connect the laryngoscope handle to the laryngoscope blade. A laryngoscope handle with a metal bar is shown as element 12B in FIG. 1 of U.S. Patent Publication No. 2008/0234549, entitled LARYNGOSCOPE BLADE, filed Mar. 22, 2007 which application is incorporated herein by reference. Advantageously, the tab extension 14 of the present embodiment provides for a metal-to-metal contact point between the metal bar of the laryngoscope handle and the laryngoscope blade. This reduces the potential for breakage of the heel 16 during use. Prior designs only had the plastic heel portion attached to the metal bar of the laryngoscope handle thus sometimes causing the heel 16 to break during use. Advantageously, in the present embodiments the slot in the heel 16 and the tab extension 14 match to provide a reinforced connection point to the metal bar of the laryngoscope handle, thus making the laryngoscope blade less susceptible to breaking away during use.

The tab extension 14 of the blade 10 is connected to the heel 16 by the fastener 18. In some embodiments the fastener includes spring loaded ball poppets used to secure the laryngoscope blade to a laryngoscope handle. The fastener 18 includes a threaded portion that engages with a threaded hole in the tab extension 14 through a hole in the heel 32 (shown in FIG. 6). This configuration provides a simple means for connecting the heel 16 to the tab extension 14 while simultaneously providing a means for connecting the laryngoscope blade to the laryngoscope handle.

The laryngoscope blades come in various sizes to accommodate use for individuals of different sizes. In some embodiments, a set of disposable laryngoscope blades of varying sizes are sold or kept together and can be used with a single laryngoscope handle. In other embodiments, reusable blades can be used in place of a set of disposable laryngoscope blades of varying sizes. A larger sized blade would be used on an adult patient whereas a smaller blade size would be used on a child. Both durable and disposable blades are difficult to differentiate by size under emergency conditions and particularly in the pre-hospital care environment. In order to readily identify the size of the blade, in accordance with one embodiment, the heel 16 is made from a color coded non-metallic material (e.g., plastic or synthetic). The color coded heel 16 indicates a size of the blade 12 to medical personal or anyone else using the laryngoscope blade. For example, a first size blade is indicated by a pink colored heel while a second size is indicated by a yellow colored heal. The tab extension 14 optionally includes a hole 22 such that the color coded heel 16 is readily shown from both sides of the laryngoscope blade. In this manner, the size of the laryngoscope blade can be easily determined by medical personal from both sides of the laryngoscope blade. Optionally, as is shown in FIG. 2, the heel 16 is formed such that a portion of the heel is also visible from the top of the laryngoscope blade. In this regard, the colored heel is visible, in some embodiments, from any direction. The set of disposable blades comes with, for example, five different sized blades all with different colored heels 16. In this manner, it is important for medical personnel to quickly access the correct sized blade in an emergency situation. The color coded heels provide such guidance without the need for the medical personal to search for a size indication printed on the blade or heel, which can also optionally be included on the blade. Thus, in some embodiments, the blade 10 includes a marking on the blade portion that shows the size of the blade. This marking can include, for example, a number indicating the size of the blade.

Referring to FIG. 6, a side cross sectional view of the laryngoscope blade of FIG. 1 is shown in accordance with one embodiment. Shown are the top portion of the blade 12, the heel 16, the lamp 20, the conduit 24, the metal contact 26, a threaded portion 36 of the conduit 24, a threaded hole 38 of the heel 16, and a wire 30. In the present embodiment, the metal contact 26 is engaged with the heel 16 through a friction fit. The metal contact 26 is coupled to the wire which goes through the conduit 24 and is coupled to the lamp 20. The conduit 24 includes the threaded portion which engages with a threaded hole 38 in the heel 16. In this manner, the conduit is securely fastened to the heel 16 eliminating any need for adhesives, brazing or welding operations in order to provide a stable conduit 24.

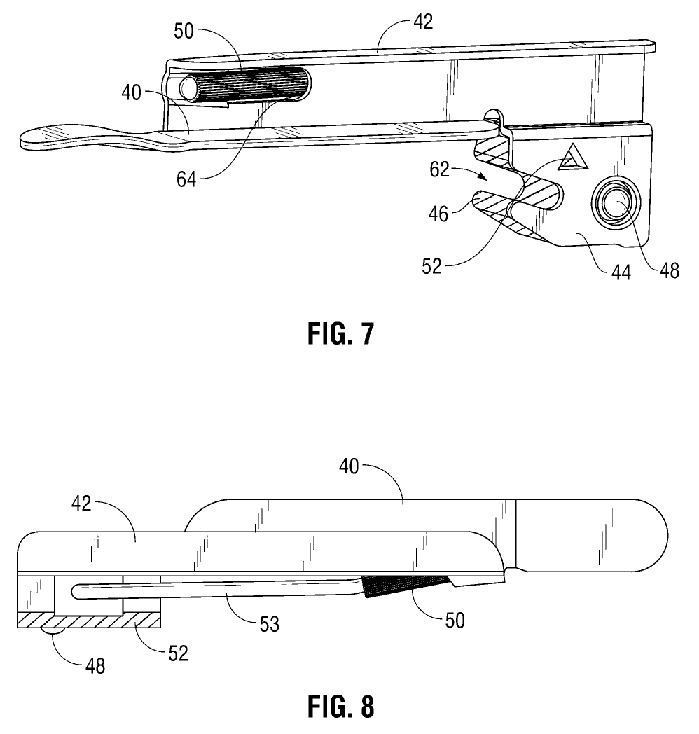

Referring to FIG. 7 a three dimensional diagram is shown illustrating a laryngoscope blade in accordance with another embodiment. The laryngoscope blade shown in FIG. 7 is known as a “Miller” blade. FIGS. 8-11 show various different views of the laryngoscope blade of FIG. 7. FIG. 12 is a side cross sectional view of the laryngoscope blade of FIG. 7 showing a threaded heel section in accordance with one embodiment. Shown are a blade 40, a top portion of the blade 42, a tab extension 44 of the blade, a heel 46, a fastener 48, a lamp 50, a hole 52 in the tab extension 44 of the blade, and a slot 62 in the tab extension 44 of the blade and a slot 64 in the blade 40. Shown in FIGS. 7-10 are a conduit 53 and a metal contact 54. Shown in FIG. 12 is a threaded portion 56 of the conduit 53, a threaded hole 57 of the heel 46, a wire 58, and a hole 60 in the heel 46. The laryngoscope blade shown in FIGS. 7-12 operates in substantially the same manner as the laryngoscope blade shown in FIGS. 1-6.

The laryngoscope blade shown in this disclosure is a single use laryngoscope blade that in use attaches to a laryngoscope handle (not shown). However, many, or all, of the features of the present embodiments can also be applied to a reusable laryngoscope blade.

While the invention herein disclosed has been described by means of specific embodiments and applications thereof, other modifications, variations, and arrangements of the present invention may be made in accordance with the above teachings other than as specifically described to practice the invention within the spirit and scope defined by the following claims.

We claim:

1. A laryngoscope blade comprising:
a blade portion including a tab extension, the tab extension including a tab extension slot for receiving a portion of a laryngoscope handle; and
a heel coupled to the tab extension by means of a fastener, the heel including a heel slot that aligns with the tab extension slot and also receives the portion of the laryngoscope handle, wherein the heel is colored to indicate a size of the blade portion of the laryngoscope blade.

2. The laryngoscope blade of claim 1 further comprising:
a conduit coupled to the heel, the conduit including a threaded portion that engages a threaded hole in the heel; and
a lamp coupled to the conduit.

3. The laryngoscope blade of claim 2 further comprising:
a metal contact coupled to the heel; and
a wire contained at least partially within the conduit and coupled to the metal contact at a first end and coupled to the lamp at a second end.

4. The laryngoscope blade of claim 3 wherein the conduit is located on a first side of the blade portion and the lamp is positioned through a slot in the blade portion such that the distal end of the lamp is located on a second side of the blade portion of the laryngoscope blade.

5. The laryngoscope blade of claim 1 further comprising a hole in the tab extension of the blade such that the heel is visible through the hole.

6. The laryngoscope blade of claim 1 wherein the fastener is attached to the tab extension by means of a threaded hole in the tab extension.

7. The laryngoscope blade of claim 1 wherein the fastener includes spring loaded ball poppets used to connect the laryngoscope blade to a laryngoscope handle.

8. The laryngoscope blade of claim 1 wherein the blade and tab extension are formed from a single piece of metal.

9. The laryngoscope blade of claim 1 wherein the laryngoscope blade is a disposable blade.

10. A set of blades comprising:
a plurality of laryngoscope blades each including a blade portion and a tab extension, the tab extension including a tab extension slot for receiving a portion of a laryngoscope handle, wherein the plurality of laryngoscope blades includes blades of at least two different sizes; and
a plurality of heels coupled to the tab extension of each of the plurality of laryngoscope blades by means of a fastener, the heel of each of the plurality of laryngoscope blades including a heel slot that aligns with the tab extension slot, the heel slot receiving the portion of the laryngoscope handle, wherein the plurality of heels are colored to indicate the size of the laryngoscope blades.

11. The set of blades of claim 10 further comprising:
a conduit coupled to the heel, the conduit including a threaded portion that engages a threaded hole in the heel; and
a lamp coupled to the conduit.

12. The set of blades of claim 11 further comprising:
a metal contact coupled to the heel; and
a wire contained at least partially within the conduit and coupled to the metal contact at a first end and coupled to the lamp at a second end.

13. The set of blades of claim 12 wherein the conduit is located on a first side of the blade portion and the lamp is positioned through a slot in the blade portion such that the distal end of the lamp is located on a second side of the blade portion of the laryngoscope blade.

14. The set of blades of claim 10 further comprising a hole in the tab extension of the blade such that the heel is visible through the hole.

15. The set of blades of claim 10 wherein the fastener is attached to the tab extension by means of a threaded hole in the tab extension.

16. The set of blades of claim 10 wherein the fastener includes spring loaded ball poppets used to connect the laryngoscope blade to a laryngoscope handle.

17. The set of blades of claim 10 wherein the blade and tab extension are formed from a single piece of metal.

18. The set of blades of claim 10 wherein the plurality of laryngoscope blades are disposable blades.
